(19) United States
(12) Patent Application Publication
(10) Pub. No.: US 2024/0008987 A1
(43) Pub. Date: Jan. 11, 2024

(54) TISSUE-CONTRACTING IMPLANTS

(71) Applicant: Edwards Lifesciences Innovation (Israel) Ltd., Caesarea (IL)

(72) Inventors: Mauricio Guerrero, Hawthorne, CA (US); Meena Francis, Trabuco Canyon, CA (US); David M. Taylor, Lake Forest, CA (US); Lindsay Marie Hall, Murietta, GA (US)

(21) Appl. No.: 18/472,162

(22) Filed: Sep. 21, 2023

Related U.S. Application Data
(63) Continuation of application No. PCT/IB2022/052523, filed on Mar. 21, 2022.
(60) Provisional application No. 63/166,170, filed on Mar. 25, 2021.

(51) Int. Cl. A61F 2/24

(52) U.S. Cl. CPC A61F 2/2466; A61F 2/2451; A61F 2220/0016

(57) ABSTRACT

Systems for use at a valve disposed between an atrium and a ventricle of a heart of a subject comprise an implant comprising a first anchoring assembly and a second anchoring assembly, each of the anchoring assemblies comprising multiple anchors, and a connector, connecting the multiple anchors to each other. A bridge couples the first anchoring assembly to the second anchoring assembly, and has an adjustable bridging length. A driver is transluminally advanceable to the atrium, and is configured to use the anchors of the respective anchoring assembly to anchor each of the respective anchoring assemblies to an opposing side of the valve, such that the bridge spans at least partway across the valve. The distance between the first and second anchoring assemblies is adjustable using a tool to adjust the bridging length of the bridge. Other embodiments are also described.

### CROSS-REFERENCES TO RELATED APPLICATIONS

The present application is a Continuation of International Patent Application PCT/IB2022/052523 to Guerrero et al., filed Mar. 21, 2022, which published as WO 2022/200972, which claims priority to U.S. Provisional Patent Application 63/166,170 to Guerrero et al., filed Mar. 25, 2021, and entitled “Tissue-contracting implants.”

Each of the above references is incorporated herein by reference.

### BACKGROUND

Annuloplasty involves remodeling tissue of an annulus of a heart valve. This can be done by pulling tissue about the annulus to a new shape. Tissue anchors can be used to facilitate medical procedures including annuloplasty, other remodeling of tissues, and securing implants. In some instances, tissue anchors can be used as an alternative to sutures. For example, tissue anchors may be used to implant an annuloplasty ring or band that extends around at least part of the annulus.

### SUMMARY OF THE INVENTION

Some of the concepts and applications thereof disclosed herein relate to implants for use at an atrioventricular valve (e.g., a mitral valve or a tricuspid valve) of a subject. Such implants can include at least two anchoring assemblies, coupled to each other by bridges that have adjustable bridging lengths. Each such anchoring assembly can include multiple anchors connected by a connector, and is configured to be anchored by the anchors to a respective portion of tissue around the valve (e.g., a respective portion of the valve annulus, such as a respective side of the valve annulus), such that subsequent adjustment (e.g., reduction) of the bridging length reshapes the valve (e.g., contracts the valve annulus).

For some applications, the bridges and connectors include (e.g., are define by) various portions of tethers that are slidably coupled to the anchors. For such applications, the bridging length can be adjusted by adjusting tension on the tethers.

For some applications, the implant includes an adjustment mechanism that is operatively coupled to one or more of the bridges. For such applications, the bridging length can be adjusted by actuating the adjustment mechanism.

The implant can be advanced to the heart transluminally and/or transcatheterally. For some applications, the implant is advanced to the heart in a preassembled state. For some applications, the implant is assembled intracardially.

Often, a driver is used to anchor the anchors to the tissue. Once the implant has been implanted, each of the bridges often spans at least partway across the valve (e.g., within the atrium, across the orifice and/or leaflets of the valve).

Adjustment of the bridging length can be performed using a tool that is advanced to the implant in the heart.

There is provided, in accordance with some applications, a system for use at a valve disposed between an atrium and a ventricle of a heart of a subject. The system includes an implant, a driver, a tool, and often also a catheter that is transluminally advanceable to the heart. In some applications, the implant includes (a) a first anchoring assembly and a second anchoring assembly, each including multiple anchors and a connector that connects the multiple anchors to each other, and (b) one or more bridges, coupling the first anchoring assembly to the second anchoring assembly, and having an adjustable bridging length.

In some applications, the driver can be extendable through the catheter, and is further often configured to use the anchors of the first anchoring assembly to anchor the first anchoring assembly, within the atrium, at a first portion (e.g., a first side) of the valve, and to use the anchors of the second anchoring assembly to anchor the second anchoring assembly, within the atrium, at a second side of the valve, such that each of the bridges spans at least partway across the valve. The tool is configured to adjust a distance between the first anchoring assembly and the second anchoring assembly by adjusting the bridging length of at least one of the bridges.

There is provided, in accordance with some applications, a method for use at the valve. In the atrium, a first series of anchors of a first anchoring assembly is anchored in a first row (e.g., in a generally straight line or in an arc) along a first portion of the annulus—e.g., such that a first connector of the first anchoring assembly connects the anchors of the first series to each other and extends along the first portion of the annulus. Also in the atrium, a second series of anchors of a second anchoring assembly is anchored in a second row along a second portion of the annulus, e.g., such that a second connector of the second anchoring assembly connects the anchors of the second series to each other and extends along the second portion of the annulus.

Typically, a tool is subsequently transluminally advanced to the heart. A distal end of the tool can be positioned within the atrium, e.g., over the orifice and/or leaflets of the valve. The tool can be used to reshape the annulus, e.g., by reducing the bridging length of a bridge that couples the first anchoring assembly to the second anchoring assembly.

The above method(s) can be performed on a living animal or on a simulation, such as on a cadaver, cadaver heart, simulator (e.g., with the body parts, heart, tissue, etc. being simulated), etc.

There is provided, in accordance with some applications, a system and/or an apparatus for use at a valve disposed between an atrium and a ventricle of a heart of a subject, the system and/or apparatus including a catheter, a first subassembly, a second subassembly, and a tool. The catheter is transluminally advanceable to the heart. The first subassembly can include (i) a first tether, having a first end portion, a second end portion, and a first bight therebetween, and being advanceable, bight-first, distally out of the catheter and into the heart; and (ii) a first set of anchors, configured to anchor the first bight to tissue of the heart such that the anchors of the first set are slidably coupled to the first bight in a first series.

In some applications, the second subassembly can include (i) a second tether, having a third end portion, a fourth end portion, and a second bight therebetween, and being advanceable, bight-first, distally out of the catheter and into the heart; and (ii) a second set of anchors, configured to anchor the second bight to tissue of the heart such that the anchors of the second set are slidably coupled to the second bight in a second series.

In some applications, the tool is slidable over the first, second, third, and fourth end portions toward the first and second bight. The tool can be configured to selectively apply tension to the first tether and the second tether by selectively pulling each of the first, second, third, and fourth end portions.

In some applications, the system and/or apparatus further includes a driver, configured to anchor each anchor of the first set to the tissue of the heart.

In some applications, the driver is configured to be transluminally advanced to the heart while coupled to a first anchor of the first set, and to anchor the first anchor to the tissue.

In some applications, the driver is configured to be transluminally advanced to the heart while the first anchor of the first set is slidably coupled to the first bight.

In some applications, the driver is configured to transluminally advance the first bight through the catheter to the heart by transluminally advancing the first anchor of the first set through the catheter while the first anchor of the first set is slidably coupled to the first bight.

In some applications, the driver is configured to slide a second anchor of the first set over and along the first end portion to the first bight, and to subsequently anchor the second anchor to the tissue, thereby arranging at least part of the first bight along the tissue between the first and second anchors of the first set.

In some applications, the driver is configured to anchor, in the atrium: each anchor of the first set in a first row along the tissue of the heart, thereby arranging at least part of the first bight along the tissue, and each anchor of the second set in a second row along the tissue of the heart, the second row being across the valve from the first row, thereby arranging at least part of the second bight along the tissue across the valve from the first bight.

In some applications, once the anchors of the first set are anchored in the first row and the anchors of the second set are anchored in the second row, the tool is configured such that sliding of the tool over the first, second, third, and fourth end portions draws the first, second, third, and fourth end portions to converge at a bridging node partway between the first row and the second row.

In some applications, the tool is configured to advance a lock over the first, second, third, and fourth end portions toward the first and second bights, and to lock the tension in the first tether and the second tether by locking the lock to the first, second, third, and fourth end portions.

In some applications, the valve has an annulus that defines an orifice, and the tool is configured to lock the lock to the first, second, third, and fourth end portions such that the lock is positioned over the orifice.

In some applications, the tool has an extracorporeal proximal portion that includes a user interface including: a first tether controller, configured to pull on the first end portion; a second tether controller, configured to pull on the second end portion; a third tether controller, configured to pull on the third end portion; and a fourth tether controller, configured to pull on the fourth end portion.

In some applications, the tool includes at least one engagement controller, configured, subsequently to the advancement of the tool over the first, second, third, and fourth end portions, to engage: the first tether controller to the first end portion, the second tether controller to the second end portion, the third tether controller to the third end portion, and the fourth tether controller to the fourth end portion.

There is provided, in accordance with some applications, a method for use at a valve disposed between an atrium and a ventricle of a heart of a subject, the valve defining an orifice and having an annulus that circumscribes the orifice, and the method including: (i) in the atrium, arranging a bight of a first tether along a first portion of the annulus by anchoring, to the first portion of the annulus, a first series of anchors that are slidably coupled to the first bight, such that a first end portion and a second end portion of the first tether are disposed outside of the heart, the first bight and the first series defining a first anchoring assembly; and (ii) in the atrium, arranging a bight of a second tether along a second portion of the annulus by anchoring, to the second portion of the annulus, a second series of anchors that are slidably coupled to the second bight, such that a third end portion and a fourth end portion of the second tether are disposed outside of the heart, the second bight and the second series defining a second anchoring assembly.

The method can further include subsequently transluminally advancing a tool over and along the first, second, third, and fourth end portions and into the atrium, in a manner that defines: (i) at a distal end of the tool, a bridging node, (ii) a part of the first tether as a first bridging portion, extending between the first anchoring assembly and the bridging node, (iii) another part of the first tether as a second bridging portion, extending between the first anchoring assembly and the bridging node, (iv) a part of the second tether as a third bridging portion, extending between the second anchoring assembly and the bridging node, and (v) another part of the second tether as a fourth bridging portion, extending between the second anchoring assembly and the bridging node, the first, second, third, and fourth bridging portions converging at the bridging node.

The method can further include subsequently reshaping the annulus by drawing at least part of the first anchoring assembly closer to the second anchoring assembly by applying tension to the first tether and to the second tether. The tension can be subsequently locked in the first tether and the second tether by locking a lock to the first tether and the second tether at the bridging node.

In some applications, the first series of anchors is a first series of anchors that are threaded onto the first bight, and anchoring the first series of anchors to the first portion of the annulus includes anchoring, to the first portion of the annulus, the first series of anchors that are threaded onto the first bight.

In some applications, applying tension to the first tether and to the second tether includes applying tension to the first tether independently of applying tension to the second tether.

In some applications, applying tension to the first tether and to the second tether includes pulling on the first end portion, and pulling on the third end portion independently of pulling on the first end portion.

In some applications, applying tension to the first tether and to the second tether includes pulling on the first end portion, and pulling on the second end portion independently of pulling on the first end portion.

In some applications, anchoring the first series of anchors and the second series of anchors includes anchoring the first series of anchors and the second series of anchors such that, for each of the first and second series, (i) one of the anchors of the series is a first terminal-anchor of the series, and is disposed at a first end of the series, and (ii) another of the anchors of the series is a second terminal-anchor of the series, and is disposed at a second, opposite end of the series.

In some applications, transluminally advancing the tool includes transluminally advancing the tool such that: the first bridging portion extends between the bridging node and the first terminal-anchor of the first series, the second bridging portion extends between the bridging node and the second terminal-anchor of the first series, the third bridging portion extends between the bridging node and the first terminal-anchor of the second series, and the fourth bridging portion extends between the bridging node and the second terminal-anchor of the second series.

In some applications, the first portion of the annulus is at a root of a first leaflet of the valve, and the second portion of the annulus is at a root of a second leaflet of the valve, and reshaping the annulus includes drawing the first and second leaflets toward each other.

In some applications, the valve is a mitral valve of the heart, and reshaping the annulus includes reshaping the annulus of the mitral valve.

In some applications, the valve is a tricuspid valve of the heart, and reshaping the annulus includes reshaping the annulus of the tricuspid valve.

In some applications, arranging the bight of the first tether along the first portion of the annulus includes: transluminally advancing the first bight to the atrium by transluminally advancing the first anchor of the first series to the atrium while the first anchor of the first series is slidably coupled to the first bight; subsequently anchoring the first anchor of the first series, subsequently, transluminally advancing a second anchor of the first series over and along the first end portion toward the first anchor of the first series and into the atrium, and subsequently, anchoring the second anchor of the first series to the first portion of the annulus.

In some applications, arranging the bight of the first tether along the first portion of the annulus includes: subsequently to anchoring the second anchor of the first series, transluminally advancing a third anchor of the first series over and along the first end portion toward the second anchor of the first series and into the atrium, and subsequently, anchoring the third anchor of the first series to the first portion of the annulus.

In some applications, arranging the bight of the first tether along the first portion of the annulus includes: subsequently to anchoring the second anchor of the first series, transluminally advancing a third anchor of the first series over and along the second end portion toward the second anchor of the first series and into the atrium, and subsequently, anchoring the third anchor of the first series to the first portion of the annulus.

In some applications, the method further includes withdrawing the anchor driver from the subject after anchoring the first anchor of the first series, and prior to advancing the second anchor of the first series.

In some applications, transluminally advancing the first bight to the atrium includes transluminally advancing the first bight to the atrium using an anchor driver engaged with the first anchor of the first series.

In some applications, transluminally advancing the second anchor of the first series includes transluminally advancing the second anchor of the first series using the anchor driver.

In some applications, locking the lock to the first tether and the second tether at the bridging node includes locking the lock to the first bridging portion, the second bridging portion, the third bridging portion, and the fourth bridging portion at the bridging node.

In some applications, locking the lock to the first bridging portion, the second bridging portion, the third bridging portion, and the fourth bridging portion, includes (i) locking a first locking element of the lock to the first bridging portion, and (ii) independently of locking the first locking element to the first bridging portion, locking a second locking element of the lock to the second bridging portion.

In some applications, the method further includes, subsequently to advancing the tool in the manner that defines the bridging node and the bridging portions, and prior to locking the tension in the first and second tethers, repositioning the distal end of the tool in a manner that: repositions the bridging node, lengthens at least one of the bridging portions, and shortens at least another of the bridging portions.

In some applications, repositioning the distal end of the tool includes repositioning the distal end of the tool in a manner that repositions the bridging node away from the first anchoring assembly and toward the second anchoring assembly.

In some applications, advancing the tool into the atrium in the manner that defines the bridging node includes advancing the tool in a manner that defines the bridging node in the atrium.

In some applications, advancing the tool in the manner that defines the bridging node in the atrium includes advancing the tool in a manner that defines the bridging node over the orifice of the valve.

The above method(s) can be performed on a living animal or on a simulation, such as on a cadaver, cadaver heart, simulator (e.g., with the body parts, heart, tissue, etc. being simulated), etc.

There is provided, in accordance with some applications, a system and/or an apparatus for use at a valve disposed between an atrium and a ventricle of a heart of a subject, the system and/or apparatus including: a catheter, transluminally advanceable to the heart and an implant, deployable from the catheter within the heart.

In some applications, the implant includes a first anchoring assembly and a second anchoring assembly, each of the anchoring assemblies including: multiple anchors, and a connector, connecting the multiple anchors to each other.

In some applications, the implant further includes one or more bridges, coupling the first anchoring assembly to the second anchoring assembly, and having an adjustable bridging length.

In some applications, the system and/or apparatus includes a driver, extendable through the catheter, and configured to use the anchors of the first anchoring assembly to anchor the first anchoring assembly, within the atrium, at a first side of the valve, and to use the anchors of the second anchoring assembly to anchor the second anchoring assembly, within the atrium, at a second side of the valve, such that each of the bridges spans at least partway across the valve

In some applications, the system and/or apparatus includes a tool, transluminally advanceable to the heart, and configured to adjust a distance between the first anchoring assembly and the second anchoring assembly by adjusting the bridging length of at least one of the bridges.

In some applications, the tool is transluminally advanceable to the implant, and is configured to engage the implant within the atrium.

In some applications, the valve has an annulus and leaflets, the annulus circumscribing an orifice within which the leaflets are disposed, and the tool is configured to engage the implant, within the atrium, over the orifice.

In some applications, the tool is configured to engage the implant between the first anchoring assembly and the second anchoring assembly, exclusive.

In some applications, the connector of each of the anchoring assemblies is rigid.

In some applications, the connector of each of the anchoring assemblies is flexible.

In some applications, the connector of each of the anchoring assemblies is axially contractible.

In some applications, the connector of each of the anchoring assemblies is resistant to axial contraction.

In some applications, each of the bridges is articulatably coupled to one of the first and second anchoring assemblies.

In some applications, the one or more bridges include: a first bridge, extending from a first part of the first anchoring assembly; a second bridge, extending from a second part of the first anchoring assembly; a third bridge, extending from a first part of the second anchoring assembly; and a fourth bridge, extending from a second part of the second anchoring assembly.

In some applications, the first, second, third, and fourth bridges converge at a bridging node partway between the first anchoring assembly and the second anchoring assembly.

In some applications, the implant includes a first tether that loops: from the bridging node to the first part of the first anchoring assembly, thereby defining the first bridge, from the first part of the first anchoring assembly to the second part of the first anchoring assembly, thereby defining the connector of the first anchoring assembly, and from the second part of the first anchoring assembly back to the bridging node, thereby defining the second bridge.

In some applications, the implant includes a second tether that loops: from the bridging node to the first part of the second anchoring assembly, thereby defining the third bridge, from the first part of the second anchoring assembly to the second part of second anchoring assembly, thereby defining the connector of the second anchoring assembly, and from the second part of the second anchoring assembly back to the bridging node, thereby defining the fourth bridge.

In some applications, the system and/or apparatus further includes a lock, and the tool is configured to: adjust the bridging length of the first, second, third, and fourth bridges by applying tension to the first and second tethers, and lock the tension in the first and second tethers by locking the lock to the first and second tethers.

In some applications: each of the bridges includes first and second bridge components axially slidable with respect to each other, and the implant further includes one or more adjustment mechanisms, each of the adjustment mechanisms being operatively coupled to a respective bridge of the bridges such that, for each of the adjustment mechanisms, actuation of the adjustment mechanism adjusts the bridging length of the respective bridge by sliding the first and second bridge components of the respective bridge with respect to each other.

In some applications, for each of the adjustment mechanisms, the adjustment mechanism includes a rotatable member, and the tool is configured to actuate the adjustment mechanism by rotating the rotatable member.

In some applications, for each of the bridges, the first bridge component is articulatable with respect to the second bridge component.

In some applications, the implant further includes one or more adjustment mechanisms, each of the adjustment mechanisms being operatively coupled to at least one of the bridges, such that, for each of the adjustment mechanisms, movement of an element of the adjustment mechanism along a first axis causes contraction of the at least one of the bridges along a second axis, the second axis being orthogonal to the first axis.

In some applications, for each of the adjustment mechanisms, the element of the adjustment mechanism includes a threaded bolt, and the tool is configured to cause contraction of the at least one of the bridges along the second axis by causing movement of the bolt along the first axis by rotating the bolt.

In some applications, the implant further includes one or more adjustment mechanisms, each of the adjustment mechanisms being operatively coupled, in a Scott Russell linkage, to at least one of the bridges.

In some applications, for each of the adjustment mechanisms, the adjustment mechanism includes a threaded bolt, and the tool is configured to actuate the adjustment mechanism by rotating the bolt.

There is provided, in accordance with some applications, a method for use at a valve disposed between an atrium and a ventricle of a heart of a subject, the valve defining an orifice and having an annulus that circumscribes the orifice, and the method including: in the atrium, anchoring along a first portion of the annulus a first series of anchors of a first anchoring assembly, such that a first connector of the first anchoring assembly connects the anchors of the first series to each other and extends along the first portion of the annulus.

In some applications, the method further includes: in the atrium, anchoring along a second portion of the annulus a second series of anchors of a second anchoring assembly, such that a second connector of the second anchoring assembly connects the anchors of the second series to each other and extends along the second portion of the annulus.

In some applications, the method further includes: subsequently, transluminally advancing a tool to the heart.

In some applications, the system and/or apparatus includes subsequently, within the atrium, positioning a distal end of the tool over the orifice, and using the tool to reshape the annulus by reducing a bridging length of a bridge that couples the first anchoring assembly to the second anchoring assembly.

In some applications, anchoring the first series of anchors along the first portion of the annulus includes arranging a bight of a first tether along the first portion of the annulus by anchoring the first series of anchors along the first portion of the annulus, such that the bight of the first tether defines the first connector.

In some applications, anchoring the second series of anchors along the second portion of the annulus includes arranging a bight of a second tether along the second portion of the annulus by anchoring the second series of anchors along the second portion of the annulus, such that the bight of the second tether defines the second connector.

In some applications, the bridge is a first bridge, positioning the distal end of the tool over the orifice includes, by positioning the distal end of the tool over the orifice: forming the first bridge from a first bridging-portion of the first tether, forming a second bridge from a second bridging-portion of the first tether, forming a third bridge from a third bridging-portion of the second tether, forming a fourth bridge from a fourth bridging-portion of the second tether, and defining, at the distal end of the tool, a bridging node at which the first, second, third, and fourth bridges converge.

In some applications, positioning the distal end of the tool over the orifice includes engaging the tool with an adjustment mechanism that is coupled to the bridge and that is disposed over the orifice, and reducing the bridging length of the bridge includes reducing the bridging length of the bridge by actuating the adjustment mechanism using the tool.

In some applications, the bridge includes a first bridge component and a second bridge component, and reducing the bridging length of the bridge includes axially sliding the first bridge component with respect to the second bridge component by actuating the adjustment mechanism using the tool.

The above method(s) can be performed on a living animal or on a simulation, such as on a cadaver, cadaver heart, simulator (e.g., with the body parts, heart, tissue, etc. being simulated), etc.

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

The present invention will be more fully understood from the following detailed description of applications thereof, taken together with the drawings, in which:

### BRIEF DESCRIPTION OF THE DRAWINGS

FIGS. 1A-B, 2A-D, 3A-D, 4A-B, and 5 are schematic illustrations of an example system for use at an atrioventricular valve of a heart of a subject, and example techniques for use with the system, in accordance with some applications;

FIG. 6 is a schematic illustration of an example implant, in accordance with some applications; and

FIGS. 7A-B, 8A-G, 9A-B, and 10 are schematic illustrations of an example system for use at atrioventricular valve, and example techniques for use with the system, in accordance with some applications.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Well-known features may be omitted or simplified in order not to obscure the disclosure. Furthermore, in order to avoid undue clutter from having too many reference numbers and lead lines on a particular drawing, some components will be introduced via one or more drawings and not explicitly identified in every subsequent drawing that contains that component.

Some of the concepts and applications thereof disclosed herein relate to implants for use at an atrioventricular valve (e.g., a mitral valve or a tricuspid valve) of a subject. The drawings show the mitral valve, purely as an example. Such implants can comprise two (or more) anchoring assemblies, coupled to each other by a bridge (or multiple bridges) that has an adjustable bridging length. Each such anchoring assembly comprises multiple anchors connected by a connector, and is configured to be anchored by the anchors to a respective portion of tissue around the valve (e.g., a respective portion of the valve annulus, such as a respective side of the valve annulus), such that subsequent adjustment (e.g., reduction) of the bridging length reshapes the valve (e.g., contracts the valve annulus).

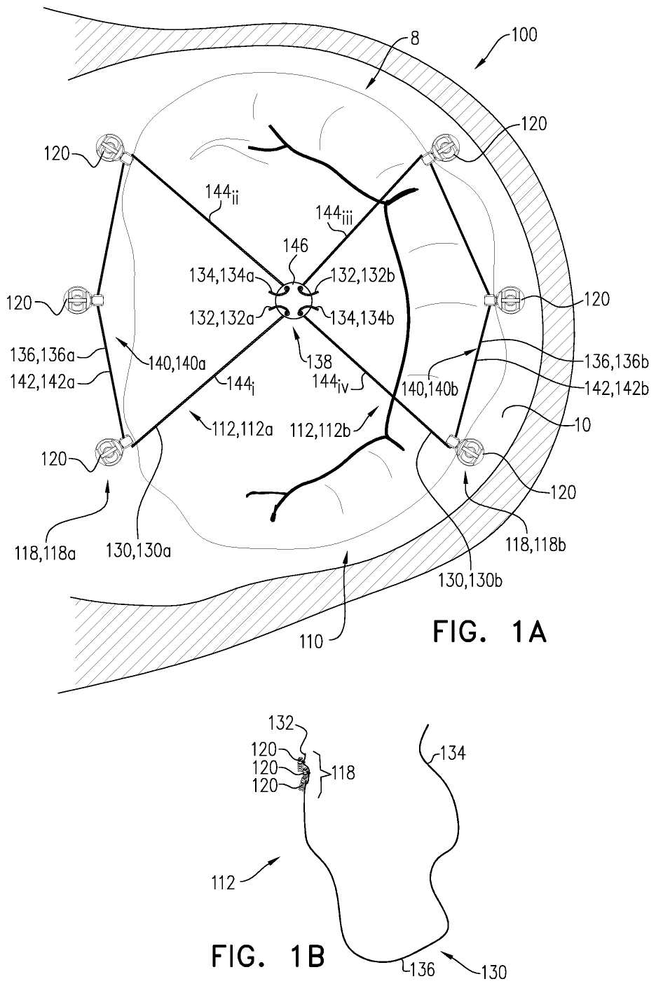

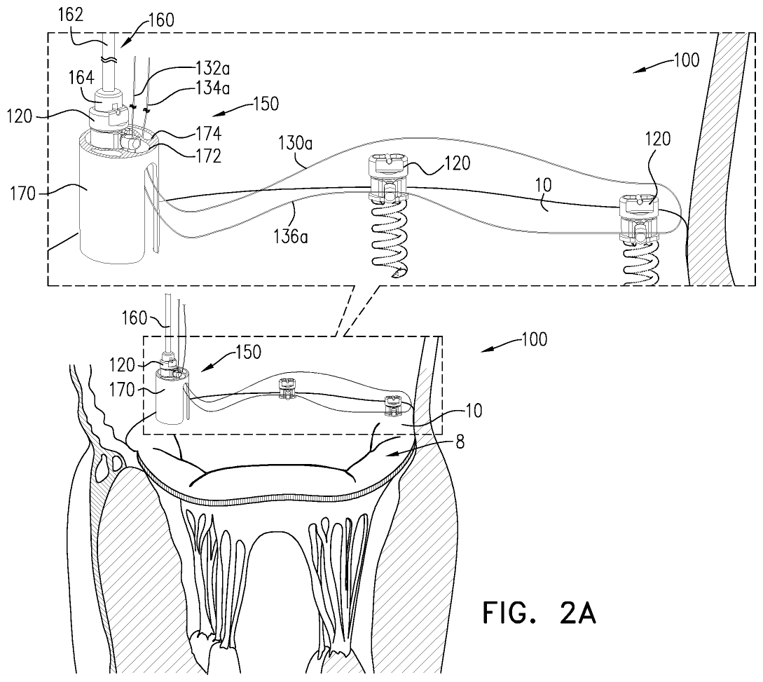

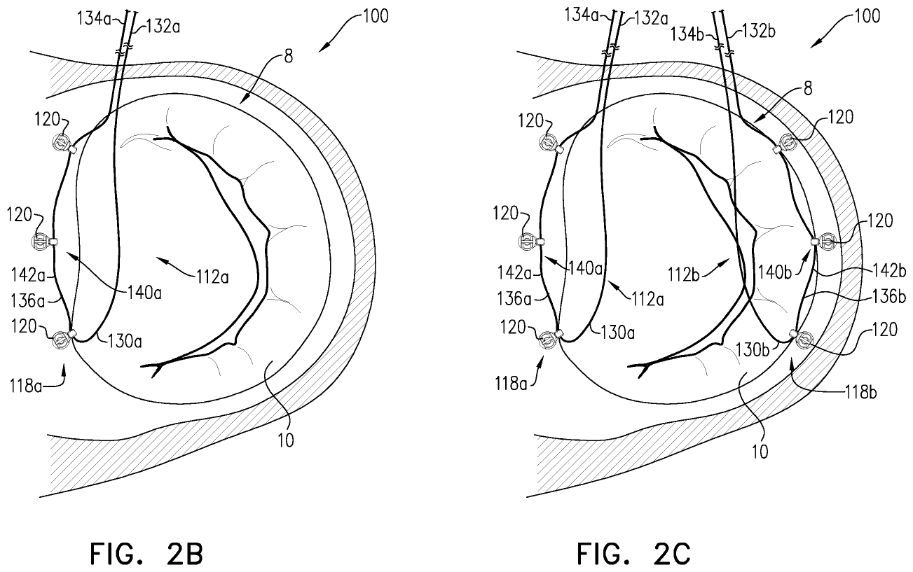

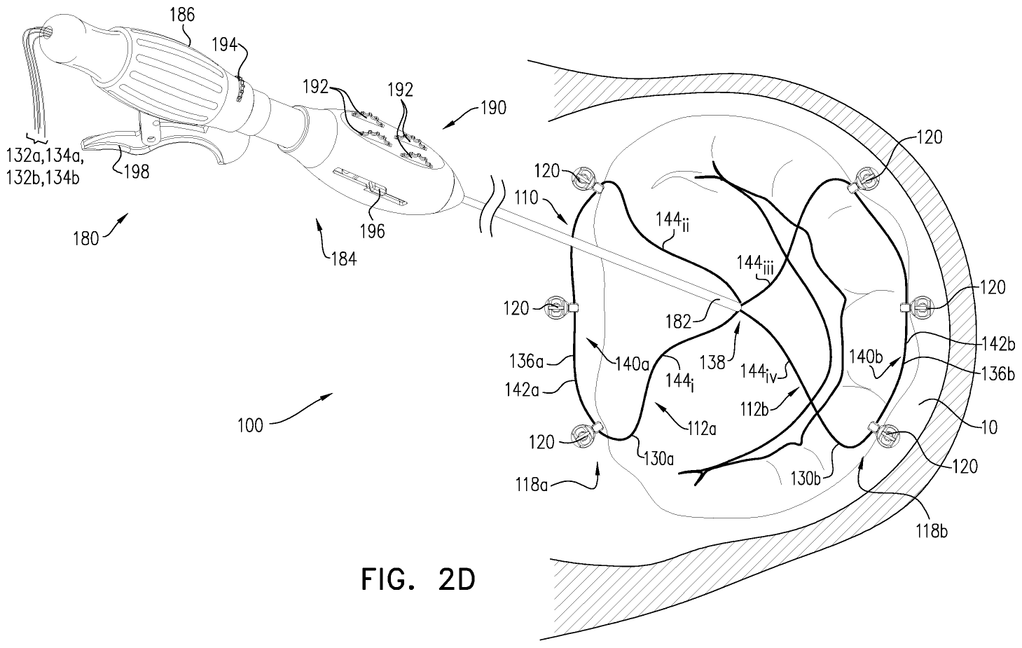

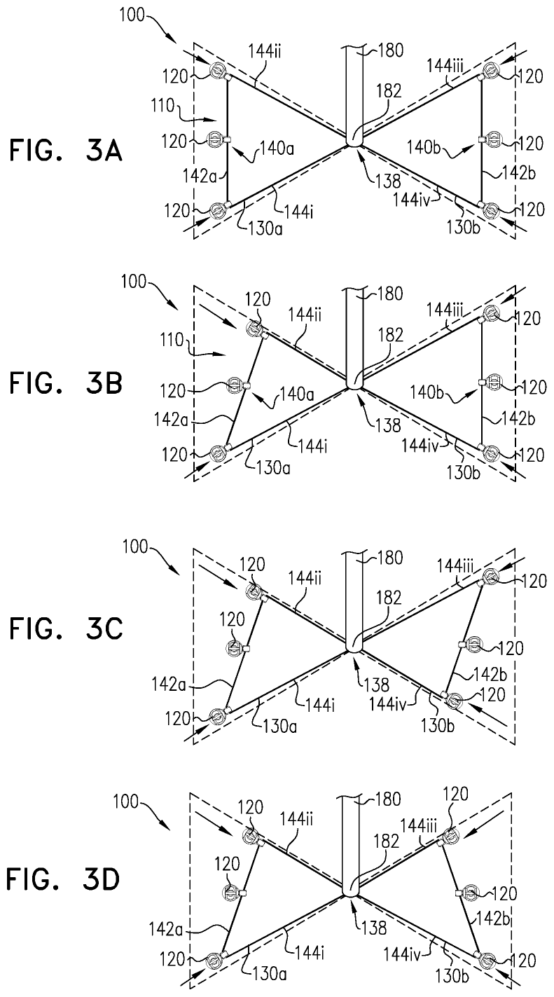

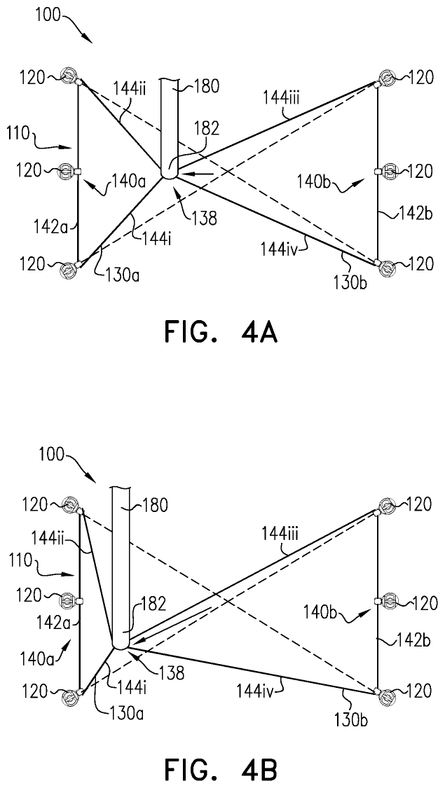

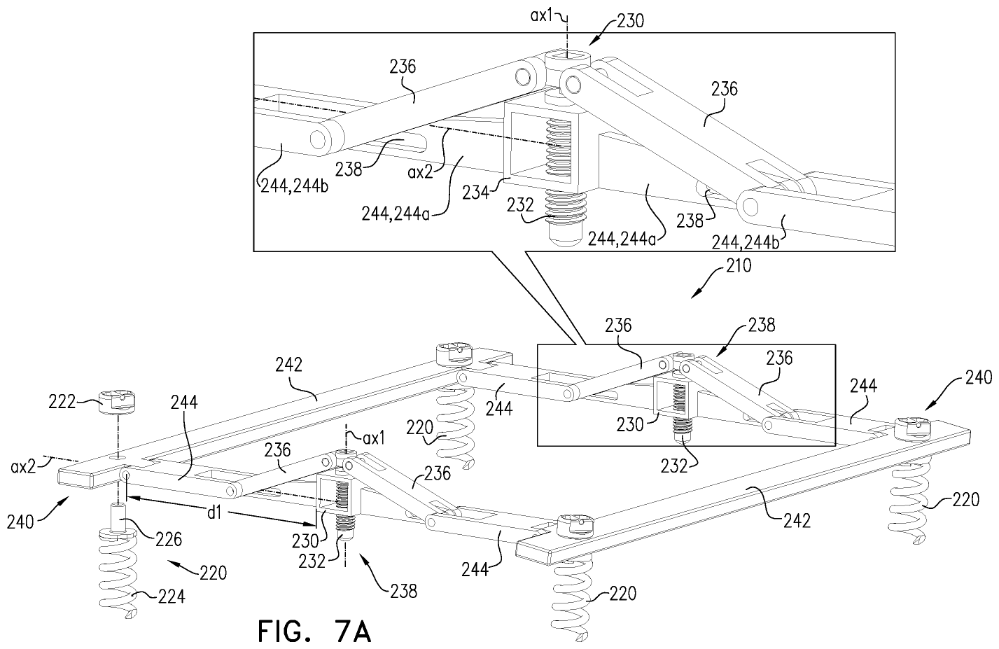

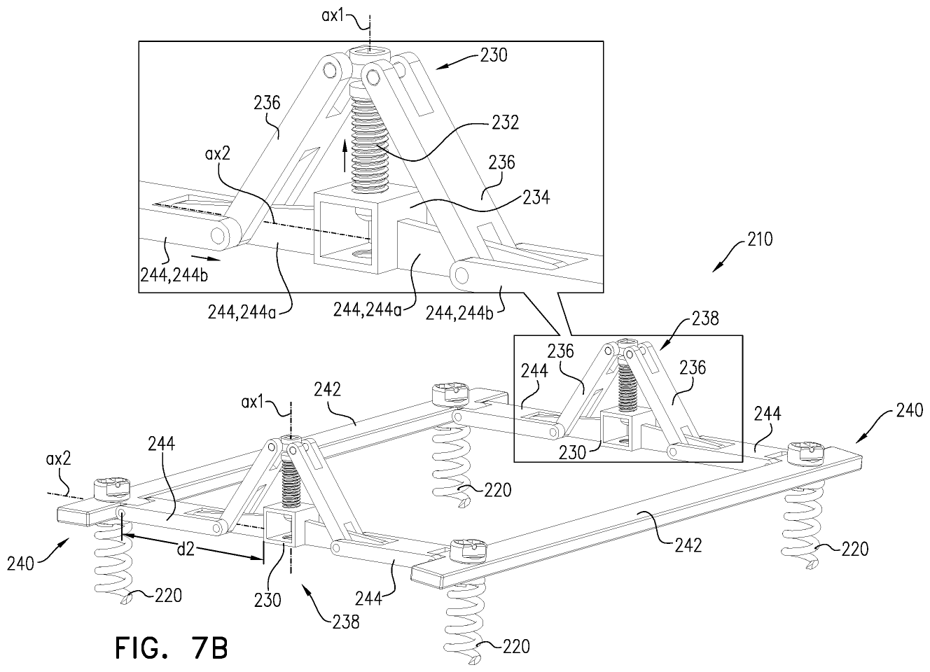

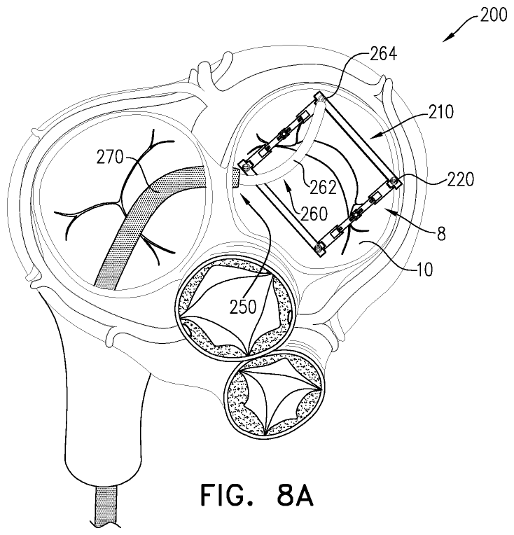

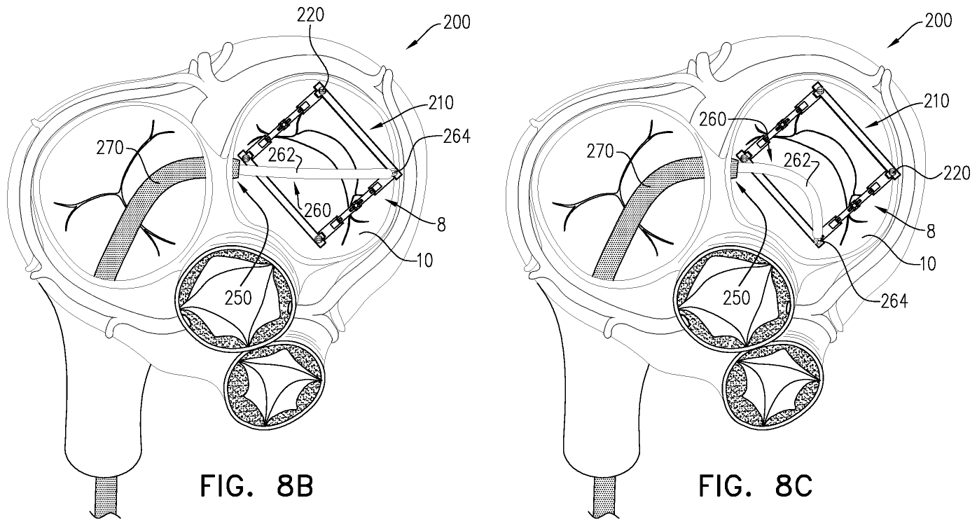

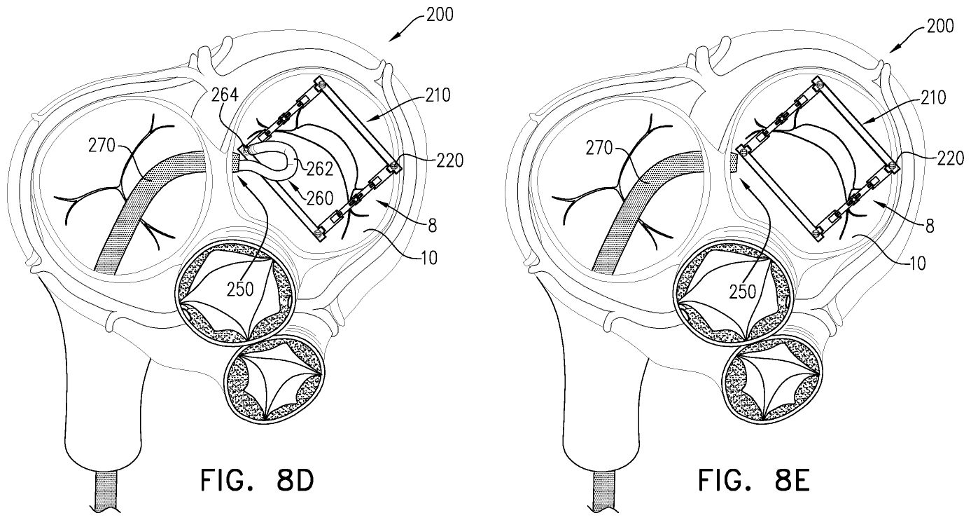

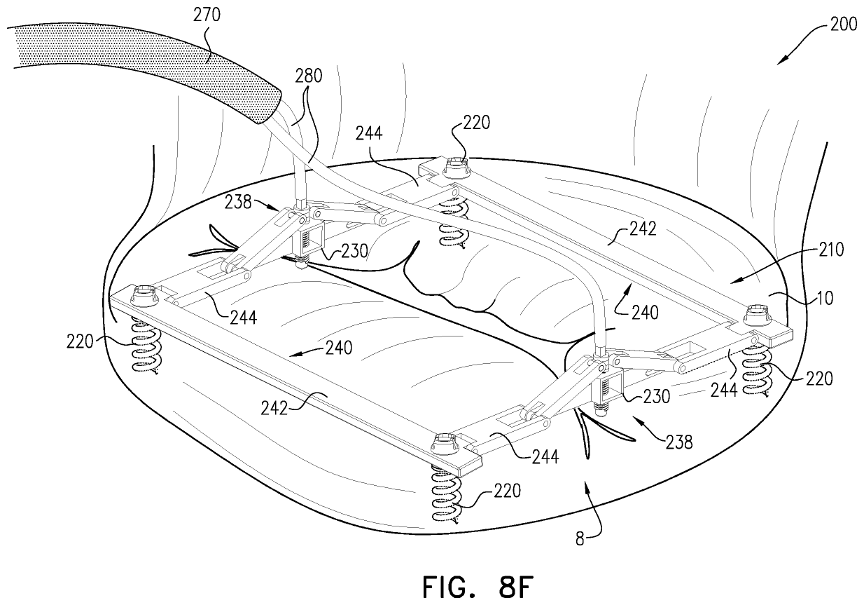

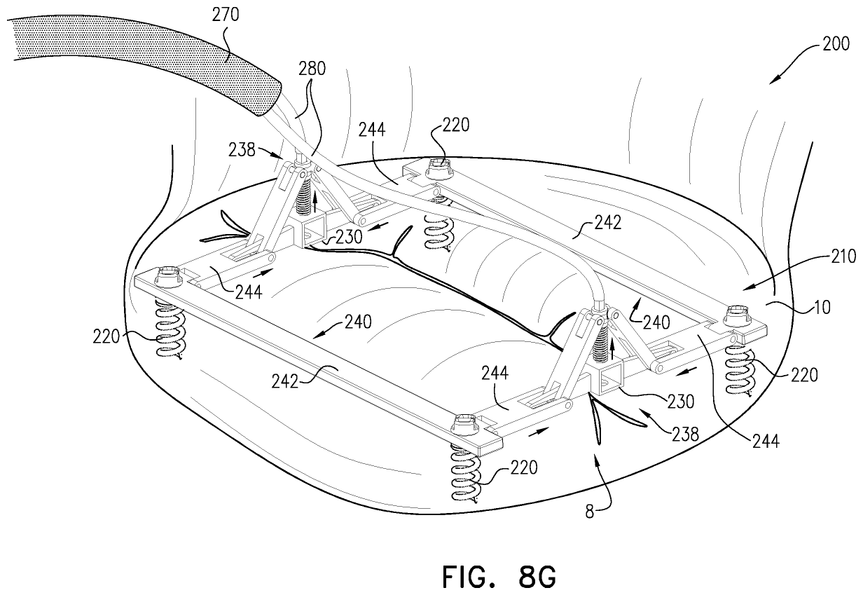

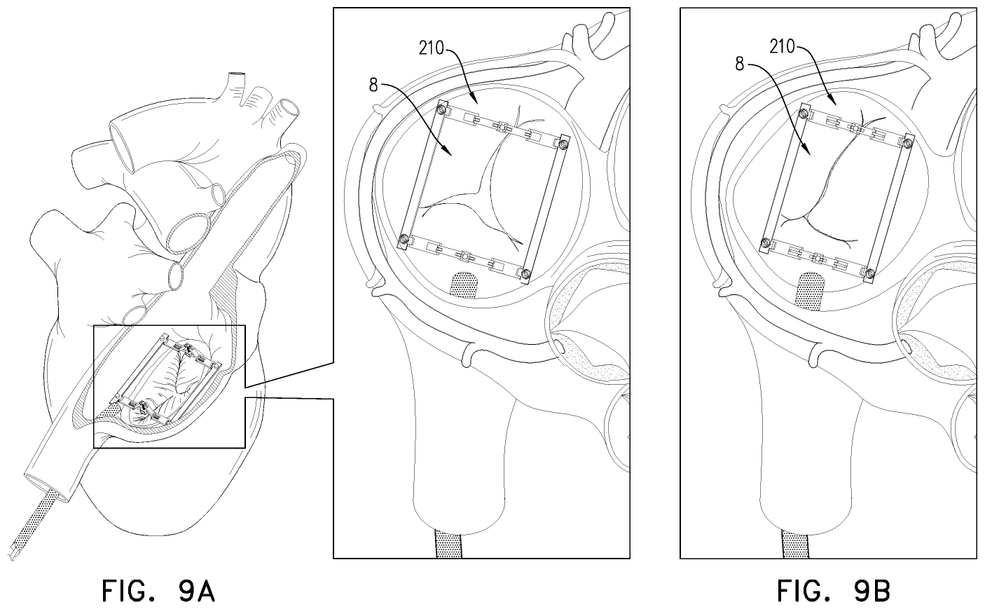

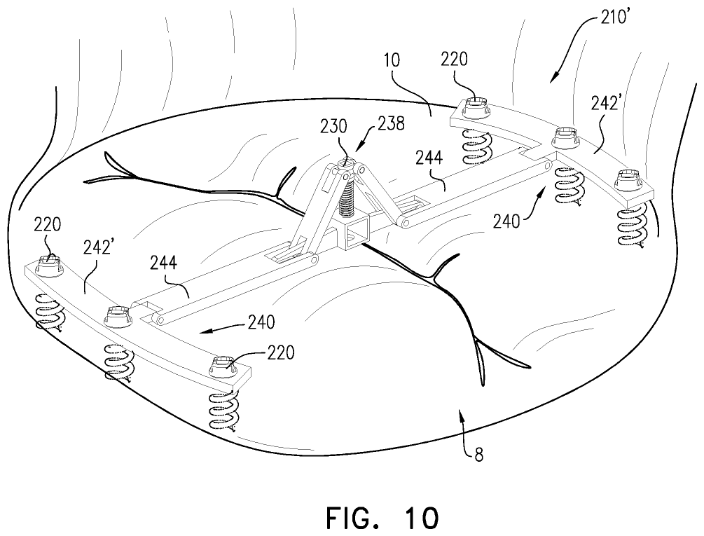

Disclosed in reference to FIGS. 1A-6 are applications in which the bridges and connectors often comprise various portions of tethers that are slidably coupled to the anchors, and in which the bridging length can be adjusted by adjusting tension on the tethers. Disclosed in reference to FIGS. 7A-10 are applications in which the implant often comprises an adjustment mechanism that is operatively coupled to the bridge, and in which the bridging length can be adjusted by actuating the adjustment mechanism. While the implants of the applications of FIGS. 7A-10 are typically advanced to the heart in a preassembled state, the implants of the applications of FIGS. 1A-6 are typically assembled intracardially.

In the applications of FIGS. 1A-6 and the applications of FIGS. 7A-10, the implant can be advanced to the heart transluminally and/or transcatheterally. In the applications of FIGS. 1A-6 and the applications of FIGS. 7A-10, a driver can be used to anchor the anchors to the tissue. In the applications of FIGS. 1A-6 and the applications of FIGS. 7A-10, once the implant has been implanted, each of the bridges typically spans at least partway across the valve (e.g., within the atrium, across the orifice and/or leaflets of the valve). In the applications of FIGS. 1A-6 and the applications of FIGS. 7A-10, adjustment of the bridging length can be performed using a tool that is advanced to the implant in the heart.

There is therefore provided, in accordance with some applications, a system for use at a valve disposed between an atrium and a ventricle of a heart of a subject. The system comprises an implant, a driver, a tool, and often also a catheter that is transluminally advanceable to the heart. The implant can comprise (a) a first anchoring assembly and a second anchoring assembly, each comprising multiple anchors and a connector that connects the multiple anchors to each other, and (b) one or more bridges, coupling the first anchoring assembly to the second anchoring assembly, and having an adjustable bridging length. The driver can be extendable through the catheter, and is further often configured to use the anchors of the first anchoring assembly to anchor the first anchoring assembly, within the atrium, at a first portion (e.g., a first side) of the valve, and to use the anchors of the second anchoring assembly to anchor the second anchoring assembly, within the atrium, at a second side of the valve, such that each of the bridges spans at least partway across the valve. The tool is configured to adjust a distance between the first anchoring assembly and the second anchoring assembly by adjusting the bridging length of at least one of the bridges.

There is further provided, in accordance with some applications, a method for use at the valve. In the atrium, a first series of anchors of a first anchoring assembly is anchored in a first row (e.g., in a generally straight line or in an arc) along a first portion of the annulus—e.g., such that a first connector of the first anchoring assembly connects the anchors of the first series to each other and extends along the first portion of the annulus. Also in the atrium, a second series of anchors of a second anchoring assembly is anchored in a second row along a second portion of the annulus, e.g., such that a second connector of the second anchoring assembly connects the anchors of the second series to each other and extends along the second portion of the annulus. Typically, a tool is subsequently transluminally advanced to the heart. A distal end of the tool can be positioned within the atrium, e.g., over the orifice and/or leaflets of the valve. The tool can be used to reshape the annulus, e.g., by reducing the bridging length of a bridge that couples the first anchoring assembly to the second anchoring assembly.

For some applications, the first portion of the annulus is at the root of a first leaflet of the valve, and the second portion of the annulus is at the root of a second leaflet of the valve, and the reshaping of the annulus draws the first and second leaflets toward each other. For example, when the mitral valve is being treated, the first anchoring assembly can be anchored to the mitral annulus at the root of the anterior mitral leaflet, the second anchoring assembly can be anchored to the mitral annulus at the root of the posterior mitral leaflet, and reshaping of the mitral annulus can draw the anterior and posterior mitral leaflets closer to each other. Similarly, when the tricuspid valve is being treated, the first anchoring assembly can be anchored to the tricuspid annulus at the root of the septal tricuspid leaflet, the second anchoring assembly can be anchored to the tricuspid annulus at the root of the posterior tricuspid leaflet, and reshaping of the tricuspid annulus can draw the septal and posterior tricuspid leaflets closer to each other, possibly functionally eliminating the anterior tricuspid leaflet. Optionally, when the tricuspid valve is being treated, the first anchoring assembly can be anchored to the tricuspid annulus at the root of the septal tricuspid leaflet, the second anchoring assembly can be anchored to the tricuspid annulus at the root of the posterior tricuspid leaflet, and a third anchoring assembly can be anchored to the tricuspid annulus at the root of the anterior tricuspid leaflet, and reshaping of the tricuspid annulus can draw the septal, posterior, and anterior tricuspid leaflets closer to each other. These method(s) can be performed on a living animal or on a simulation, such as on a cadaver, cadaver heart, simulator (e.g., with the body parts, heart, tissue, etc. being simulated), etc.

Reference is made to FIGS. 1A-B, 2A-D, 3A-D, 4A-B, and 5, which are schematic illustrations of a system 100 for use at an atrioventricular valve 8 of a heart of a subject, and techniques for use with the system, in accordance with some applications. System 100 comprises at least two subassemblies 112, and often further comprises a delivery instrument 150 that is transluminally advanceable to the heart, and that is used to facilitate implantation (and often at least some assembly) of each subassembly. As detailed further hereinbelow, subassemblies 112 are components of an implant 110, which can be assembled intracardially. Delivery instrument 150 often comprises a driver 160, and a catheter (e.g., a flexible tube) 170 within (e.g., through) which the driver is slidable.

FIG. 1A shows implant 110 in its assembled state, e.g., after implantation at valve 8. FIG. 1B shows one of subassemblies 112, e.g., prior to implantation. Each subassembly 112 comprises a respective tether 130 and a respective set 118 of anchors 120. The suffixes a and b are used herein to designate instances of a given element (e.g., otherwise-identical instances of the element) as being of one or other of the subassemblies. For example, FIG. 1A shows implant 110 comprising (i) a first subassembly 112a that comprises a tether 130a and a set 118a of anchors 120, and (ii) a second subassembly 112b that comprises a tether 130b and a set 118b of anchors 120. Each tether 130 has a first end portion 132, a second end portion 134, and at least in the assembled state of the implant 110, a bight 136 between the end portions.

As described in more detail hereinbelow, in the assembled state of implant 110 each subassembly 112 of the implant comprises (i) a respective anchoring assembly 140 that comprises multiple anchors 120 and a connector 142 that connects the multiple anchors to each other, and (ii) two bridges 144 that couple the first anchoring assembly to the second anchoring assembly. That is, subassembly 112a comprises (i) an anchoring assembly 140a that comprises multiple anchors 120 and a connector 142a, and (ii) two bridges 144_i and 144_ii; and subassembly 112b comprises (i) an anchoring assembly 140b that comprises multiple anchors 120 and a connector 142b, and (ii) two bridges 144_iii and 144_iv. As shown, connectors 142 and bridges 144 can be defined by various portions of tethers 130. Therefore, connectors 142 and bridges 144 can be flexible.

Often, and as described in more detail hereinbelow, in the assembled state of implant 110 the bridges 144 of all (e.g., both) subassemblies 112 converge at a bridging node 138, where they are fixedly coupled to each other by a lock 146.

For completeness, in FIG. 1A, the reference numeral of each element of each subassembly is duplicated with and without its suffix, with a comma separating the non-suffixed and the suffixed reference numerals. For example, tether 130a is the tether 130 of subassembly 112a, and is therefore labeled “130, 130a”. However, to avoid crowding of the figures, FIGS. 2A-5 use only the suffixed reference numerals. (The suffixes are omitted from FIG. 1B, which shows a subassembly 112 that could be either subassembly 112a or 112b.)

At least in the assembled state of implant 110, for each subassembly 112, anchors 120 are slidably coupled to tether 130—often to bight 136. This slidable coupling can be achieved, for example, by the anchors being threaded onto tether 130. For some applications, anchors 120 and/or their slidable coupling to tether 130 are as described, mutatis mutandis, for one or more of the anchors and/or tethers described in U.S. patent application Ser. No. 17/145,258 to Kasher et al., and/or U.S. Patent Application 63/162,443 to Shafigh et al., filed Mar. 17, 2021, each of which is incorporated herein by reference for all purposes.

Driver 160 often comprises a flexible stem 162, and a driver head 164 that is configured to reversibly engage a driver interface (e.g., a head) of anchor 120. Via this engagement, driver 160 is configured to drive a tissue-engaging element of anchor 120 into tissue 10. For example, for applications in which the tissue-engaging element is helical, driver 160 can be configured to screw the tissue-engaging element into the tissue.

FIG. 2A shows bight 136a of tether 130a having been advanced distally out of catheter 170, two anchors 120 of set 118a having been anchored to tissue 10 of the heart, and driver 160 advancing a third anchor of the set through the catheter and driving the third anchor into the tissue. For some applications, bight 136a is advanced through and/or out of catheter 170 with the first anchor of set 118a already slidably coupled to tether 130a. For example, bight 136a can be advanced through and/or out of catheter 170 by driver 160 with the first anchor 120 slidably coupled to the bight, and with the driver engaged with the first anchor—e.g., the driver advances the bight by advancing the anchor.

Once the first anchor 120 of set 118a has been anchored, end portions 132a and 134a of tether 130a are disposed proximally from bight 136a (e.g., outside of the heart), typically extending transluminally out of the subject. For some applications, each subsequent anchor 120 is advanced to the heart (e.g., to the atrium) by sliding the anchor over and along one of the end portions (in the application shown, end portion 132a) toward the first anchor. Often, after the anchoring of each anchor, driver 160 is withdrawn from the heart (e.g., from the body of the subject), where it is coupled to the subsequent anchor.

For example, end portion 132a can extend through a first lumen 172 of catheter 170 (e.g., a primary lumen), and end portion 134a can extend through a second lumen 174 of the catheter (e.g., a secondary lumen). As shown, lumen 172 can be dimensioned to facilitate passage of anchors 120 and driver 160 therethrough, and lumen 174 can be narrower and/or have a smaller cross-sectional area than lumen 172.

Optionally, system 100 can be configured to facilitate advancement of the subsequent anchors over either end portion of tether 130—for example by having each end portion disposed or positionable within a lumen (e.g., a respective lumen, or the same lumen) that is dimensioned to facilitate passage of anchors 120 and driver 160 therethrough. It is hypothesized that such a configuration can advantageously allow the first anchor of the set to be anchored in a position that has been determined as being an optimal center of the tissue site/anchoring assembly 140, with subsequent anchors of the set being added on either side thereof (e.g., in an alternating manner). It is further hypothesized that such a configuration can advantageously allow the physician to make intra-procedural decisions regarding the bounds of anchoring assembly 140, such as in response to information obtained during the procedure. For example, the physician can initially intend for the first anchor of the set to be a terminal-anchor (see below), but can subsequently decide to anchor a subsequent anchor of the set beyond the first anchor.

Components and/or features of delivery instrument 150 (e.g., catheter 170 and/or driver 160) and/or anchors 120 can share one or more structural and/or functional characteristics described in one or more of the following references (e.g., structural and/or functional characteristics of one or more of the catheters, drivers, and/or anchors described therein), each of which is incorporated herein by reference:
US Patent Application Publication 2018/0049875 to Iflah et al.
PCT Application publication WO/2020/240282 to Brauon et al.
U.S. patent application Ser. No. 17/145,258 to Kasher et al.

FIGS. 2B-D are schematic top views—e.g., looking down at valve 8 from the atrium upstream of the valve, similar to the view in FIG. 1A.

FIG. 2B shows assembly/implantation of subassembly 112a (e.g., anchoring of anchors 120 of set 118a) as complete. Subassembly 112b can be assembled/implanted in the same manner, mutatis mutandis. FIG. 2C shows both subassemblies having been assembled/implanted. In the example shown, each anchor 120 of set 118a has been anchored in a first row along the tissue of the heart, thereby arranging at least part of bight 136a along the tissue, and each anchor of set 118b has been anchored in a second row along the tissue of the heart, across valve 8 from the first row, thereby arranging at least part of bight 136b along the tissue across the valve from bight 136a.

As shown, once anchors 120 of each set have been anchored, end portions 132a and 134a of tether 130a, and end portions 132b and 134b of tether 130b extend out of the heart, and typically out of the subject.

The number of anchors 120 in each set 118 can be predetermined or can be decided in real-time by the physician. Although the illustrated examples show sets 118 having an equal number of anchors 120, unequal numbers can be used. Similarly, although implant 110 is shown and described as comprising two subassemblies, a greater number of subassemblies (e.g., three subassemblies or four subassemblies) can be used, mutatis mutandis.

Once both subassemblies have been assembled/implanted, a tool 180 is subsequently transluminally advanced to the heart, often by transluminally advancing the tool over and along end portions 132a, 134a, 132b, and 134b, e.g., until a distal end 182 of the tool is positioned within the atrium, such as over the orifice and/or leaflets of valve 8 (FIG. 2D). That is, tool 180 typically engages implant 110 within the atrium. Often, this engagement occurs between anchoring assembly 140a and anchoring assembly 140b, exclusive. Often, this advancement of tool 180 defines, at distal end 182 of the tool, a bridging node 138 at which tethers 130a and 130b (e.g., end portions 132a, 134a, 132b, and 134b thereof) converge. Bridging node 138 can be partway between the row of anchors of set 118a, and the row of anchors of set 118b—e.g., over the upstream side of the orifice and/or a leaflet of valve 8.

At least in the implanted state of implant 110, the implant comprises (i) first and second anchoring assemblies 140a and 140b, each of which comprises multiple anchors 120 and a connector 142 that connects the multiple anchors to each other, and (ii) bridges 144, coupling the first anchoring assembly to the second anchoring assembly. Connectors 142 and bridges 144 of implant 110 are thereby often defined by various portions of tethers 130 and can become more distinct upon advancement of tool 180.

In some applications, and as shown in FIGS. 1A, 2D and 5, first tether 130a loops:
(i) from bridging node 138 to a first part (e.g., a first end) of first anchoring assembly 140a, thereby defining a first bridge 144_i (this portion of the tether can be referred to as a first bridging portion of the tether);
(ii) from the first part of first anchoring assembly 140a to a second part (e.g., a second end) of the first anchoring assembly, thereby defining connector 142a, and
(iii) from the second part of first anchoring assembly 140a back to bridging node 138, thereby defining a second bridge 144_ii (this portion of the tether can be referred to as a second bridging portion of the tether).

Second tether 130b similarly loops to, along, and back from second anchoring assembly 140b to define a third bridge 144_iii, connector 142b, and a fourth bridge 144_iv.

The example illustrated shows both anchoring assemblies 140 having a generally similar length to each other, both of the anchoring assemblies extending a similar distance along the valve annulus as each other, and each of the anchoring assemblies extending about a quarter of the way along the annulus. However, the anchoring assemblies can have different lengths to each other, can extend different distances along the valve annulus to each other, and each can extend more or less than a quarter of the way along the annulus.

Often, and as shown, the anchors 120 of a given set 118 are arranged in a series on their respective tether 130 and are also anchored to the tissue in the series. For each series, the anchor 120 at one end of the series can be considered a first terminal-anchor of the series, the anchor 120 at the opposite end of the series can be considered a second terminal-anchor of the series. Throughout this application (including the specification and the claims), in the context of a given series of anchors, the terms “first terminal-anchor” and “second terminal-anchor” do not necessarily indicate an order in which these two anchors are advanced and/or anchored.

For some applications, the parts of anchoring assemblies 140 from which bridges 144 extend toward bridging node 138 are the terminal-anchors. That is, for some applications, for a given subassembly 112, (i) one bridge (i.e., one bridging portion of the tether) extends between bridging node 138 and the first terminal-anchor of the respective series, and (ii) the other bridge (i.e., the other bridging portion of the tether) extends between the bridging node and the second terminal-anchor of the respective series.

For some applications, and as shown, the terminal-anchors of a given anchoring assembly 140 define the ends of that anchoring assembly.

Each bridge 144 has a bridging length between its respective anchoring assembly 140 and bridging node 138. Tool 180 is used to adjust (e.g., reduce) the bridging length of one or more of bridges 144 by applying tension to tethers 130a and 130b (e.g., by pulling on end portions 132a, 134a, 132b, and 134b). Due to the anchoring of anchors 120 to tissue 10, tissue 10 is thereby reshaped. For example, for applications in which tissue 10 is tissue of an annulus of valve 8, reduction of the bridging length reshapes (e.g., contracts) the annulus. FIG. 5 shows valve 8 after its contraction by system 100 and is discussed hereinbelow. However, beforehand, some other optional features of system 100 are described.

For some applications, each tether 130 can be tensioned independently of the other tether(s) of implant 110, or in concert with them. Furthermore, for a given tether, end portion 132 can be pulled (e.g., tensioned) independently of end portion 134, or in concert with it. For example, tool 180 can be configured to selectively or differentially apply tension to tethers 130a and 130b by selectively or differentially pulling end portions 132a, 134a, 132b, and 134b.

FIGS. 3A-D schematically represent various ways in which implant 110 can be contracted, with the broken line representing the shape of the implant (e.g., bridges 144 thereof) upon implantation, and the solid line representing the shape of the implant after contraction. FIG. 3A shows a hypothetical contracted shape of implant 110 upon all the end portions (i.e., both end portions of both tethers) being pulled in concert, resulting in a similar amount of shortening for all bridges 144. FIG. 3B shows a hypothetical contracted shape of implant 110 upon end portion 134a being pulled further than the other three end portions, resulting in bridge 144_ii being shortened more than the other three end portions. FIG. 3C shows a hypothetical contracted shape of implant 110 upon end portions 134a and 134b being pulled further than end portions 132a and 132b, resulting in bridges 144_ii and 144_iv being shortened more than bridges 144_i and 144_iii. FIG. 3D shows a hypothetical contracted shape of implant 110 upon end portions 134a and 132b being pulled further than end portions 132a and 134b, resulting in bridges 144_ii and 144_iii being shortened more than bridges 144_i and 144_iv. It is hypothesized that system 100 is thereby advantageously flexible, facilitating a high degree of control over the manner in which cardiac tissue, such as a valve annulus, is reshaped.

For some applications, the dual functions of independent pulling and in-concert pulling can be facilitated by an extracorporeal control interface 190. Interface 190 can be disposed at a proximal portion 184 of tool 180 and can be integrated with a handle 186 of the tool—e.g., as shown. Interface 190 is operable by a user such as a surgeon or interventional cardiologist.

Interface 190 can comprise a plurality of tether controllers 192, each configured to apply tension to a respective individual end portion of a tether 130 of implant 110. For example, and as shown, for applications in which implant 110 comprises tethers 130a and 130b, interface 190 can comprise four tether controllers 192—one for end portion 132a, one for end portion 134a, one for end portion 132b, one for end portion 134b. For some applications, each tether controller 192 comprises a respective wheel (as shown), knob, or similar, actuation of which pulls its respective tether end portion.

Interface 190 can comprise a collective-tether controller 194, actuation of which pulls all of the end portions collectively. For example, controller 194 can be used to achieve the result shown in FIG. 3A. Controller 194 can be used with or without use of controllers 192. For example, controller 194 might be used to achieve a certain amount of overall contraction of the annulus of valve 8, and one or more of controllers 192 might be used (before and/or afterwards) to achieve more particular changes to the shape of the annulus (e.g., as described with reference to FIGS. 3B-C).

Interface 190 can comprise an engagement controller 196, configured to reversibly engage and disengage the interface with tethers 130—e.g., to reversibly engage and disengage controllers 192 and 194 with the end portions. Controller 196 can be used, for example, to disengage interface 190 (e.g., controllers 192 and 194 thereof) from tethers 130 in order to facilitate sliding of tool 180 over and along the tethers to the heart, and to subsequently engage the interface with the tethers in order to facilitate pulling of the end portions of the tethers and contraction of implant 110.

For some applications, distal end 182, and therefore bridging node 138, can be positioned (and repositioned) at least in part independently of the locations in which anchors 120 are anchored. This positioning can be performed as distal end 182 is advanced into the heart (e.g., as the distal end is brought into proximity with valve 8), and/or can be performed once the distal end is already within the heart (e.g., after the distal end is already disposed at valve 8). This positioning can be facilitated by tensioning one or more of the end portions of tethers 130 while relaxing one or more others of the end portions of the tethers. This positioning can alternatively or additionally be facilitated by steering of distal end 182 of tool 180, and/or by steering of a catheter through which tool 180 is disposed. Such steering can be achieved by means known in the art, mutatis mutandis, such as via one or more pull-wires extending through tool 180 and/or a catheter through which the tool is disposed.

FIGS. 4A-B schematically illustrate repositioning of bridging node 138 represent various ways in which implant 110 can be contracted, with the broken line representing an initial shape of implant 110 (e.g., bridges 144 thereof) upon distal end 182 arriving at valve 8, and the solid line representing the shape of the implant after repositioning of distal end 182. The arrow indicates the resulting movement of bridging node 138 from its initial position to its subsequent position. As shown, repositioning of bridging node 138 lengthens at least one of bridging portions 144 and shortens at least another of the bridging portions. The repositioning can move distal end 182 and bridging node 138 away from one of anchoring assemblies 140 and toward another of the anchoring assemblies. FIG. 4A shows an example in which distal end 182, and therefore bridging node 138, are repositioned toward anchoring assembly 140a, lengthening bridges 144_iii and 144_iv, and shortening bridges 144_i and 144_ii. FIG. 4B shows an example in which distal end 182, and therefore bridging node 138, are repositioned toward one particular end of anchoring assembly 140a.

It is hypothesized that one or more advantages are provided by the ability to reposition bridging node 138. These advantages can be the direct result of the position of bridging node 138—e.g., by lock 146 and/or bridges 144 being disposed over the orifice and/or leaflets of valve 8, and mechanically obstructing a leaflet from flailing into the atrium.

Alternatively or additionally, these advantages can be a product of the different angles and lengths of bridges 144 resulting from the repositioning of bridging node 138 and can provide additional control over the contraction of different parts of the annulus. For example, compared to an initial central position of bridging node 138, positioning the bridging node as shown in FIG. 4B can:
increase the contraction of anchoring portion 140a caused by a given amount of pulling of end portions 132a and 134a of tether 130a (in absolute terms, and/or relative to the effect of the pulling on the bridging length of bridges 144_i and 144_ii), and/or
increase the amount by which the bridging length of bridges 144_iii and 144_iv is shortened by a given amount of pulling of end portions 132b and 134b (in absolute terms, and/or relative to the amount by which the pulling contracts anchoring portion 140b).

Once the desired adjustment (e.g., contraction) of tissue 10 (e.g., of valve 8) has been achieved, the tension that has been applied to tethers 130 is locked in place by locking lock 146 to the tethers (FIG. 5). Lock 146 can be coupled to distal end 182 of tool 180 and can be locked to the tethers by actuation of a lock controller 198 at proximal portion 184 of the tool (FIG. 5). Lock controller 198 can be a component of interface 190.

For some applications, and as shown, tool 180 is also used to cut the excess of tethers 130 proximally from lock 146. For such applications, tool 180 can be configured such that a single action by the operator (e.g., actuation of lock controller 198) both locks lock 146 and cuts the excess of the tethers.

For some applications, lock 146 and/or the locking and/or cutting operations of tool 180 can share elements and/or features with those described in US Patent Application Publication 2020/0015971 to Brauon et al., which is incorporated herein by reference.

For some applications, lock 146 comprises multiple locking elements, each locking element being independently lockable by tool 180, such that the lock is lockable to each tether 130 independently of locking to the others—e.g., such that the lock is lockable to each bridging portion (e.g., each bridge 144) independently of locking to the others.

In the application shown, these various controllers can be mechanically coupled to the elements that they control. However, alternatively or additionally, interface 190 can comprise a user interface for one or more computerized control elements that control operation of the system elements. For the purposes of this application, the term “computerized control element”, and the equivalent term “computerized controller”, refer to a computing circuit or element for controlling operation of mechanical and/or electrical components of the system. The computerized control element includes a processing unit functionally associated with a non-tangible computer readable storage medium. The storage medium stores instructions, which, when executed by the processing unit, carry out actions which control the operation of the mechanical and/or electrical components of the system. For example, the instructions can include instructions to distally advance one or more components of the system, or to proximally retract one or more components of the system. In such instances, interface 190 can be functionally associated with the one or more computerized control elements and can be configured to receive user input that thereby triggers execution of specific instructions stored in the storage medium.

Therefore, throughout this application, interface 190 can include one or more mechanical controllers such as knobs, switches, levers, sliders, and/or buttons (e.g., as shown), and/or can include one or more electronic controllers such as a keyboard, mouse, button, and/or touchscreen. Similarly, a given controller that is illustrated herein as one type of mechanical controller can optionally be provided as another type of mechanical controller.

Furthermore, it is to be noted that the shape and configuration of interface 190 (e.g., handle 186) can vary from that shown. For example, configurations are possible in which interface 190 is not integrated with a handle that is shaped to be held.

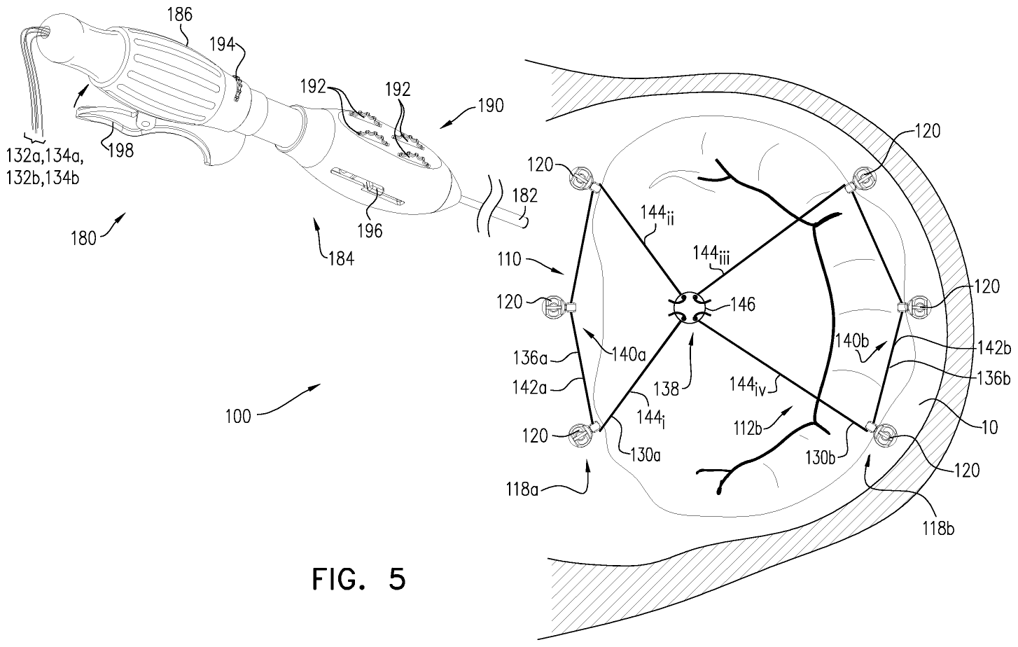

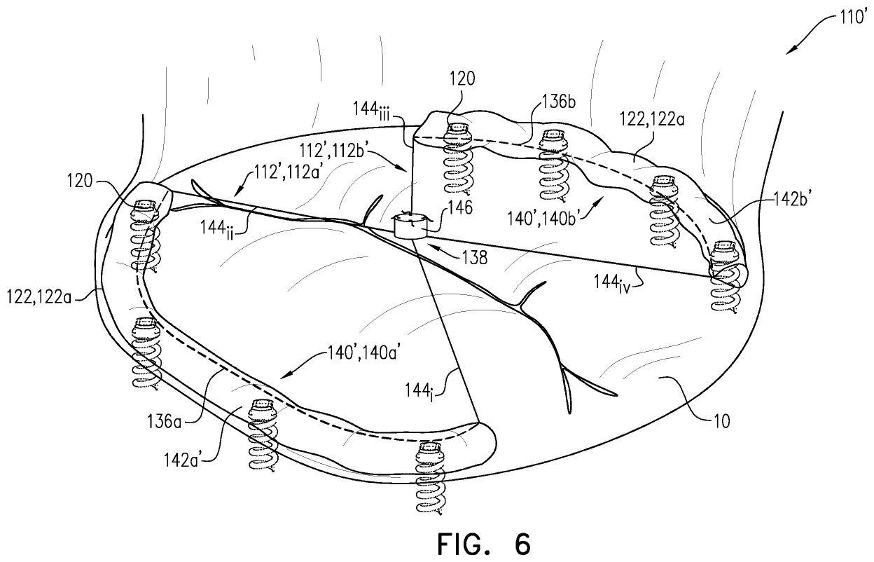

Reference is now made to FIG. 6, which is a schematic illustration of an implant 110′, in accordance with some applications. Implant 110′ is typically as described for implant 110, except where noted. Similarly, components of implant 110′ that are identically named and numbered as those of implant 110 except with the suffix ‘ are typically as described for those of implant 110, except where noted. The anchoring assembly of each subassembly of implant 110’ further comprises a sleeve 122. For example, in the application shown, anchoring assembly 140a′ of subassembly 112a′ further comprises a sleeve 122a, and anchoring assembly 140b′ of subassembly 112b′ further comprises a sleeve 122b. Sleeves 122 can be flexible and can comprise a polymer and/or a fabric.

In each subassembly 112 of implant 110′, bight 136 of tether 130 extends along sleeve 122, such as by extending through a lumen of the sleeve, or by weaving along a lateral wall of the sleeve (as shown). For some applications, anchoring assembly 140 is anchored by anchors 120 being advanced from the lumen of the sleeve, through the wall of the sleeve, and into tissue 10—e.g., similarly, mutatis mutandis, to as described in US Patent Application Publication 2012/0078355 to Zipory et al. and/or US Patent Application Publication 2015/0272734 to Sheps et al., each of which is incorporated herein by reference. For such applications, anchors 120 of implant 110′ can be slidably coupled to bight 136 by virtue of sleeve 122 being slidably coupled to the bight. For such applications, the sleeve 122 of a given anchoring assembly 140′ can define the connector 142′ of that anchoring assembly (e.g., connector 142a′ of anchoring assembly 140a′, and connector 142b′ of anchoring assembly 140b′).

For some applications, and as shown, the parts of each anchoring assembly 140a′ and 140b′ from which bridges 144 extend toward bridging node 138 are the ends of sleeves 122, which can be spaced further apart than the terminal-anchors of the anchoring assembly. That is, for some applications, for a given subassembly 112′, (i) one bridge (i.e., one bridging portion of the tether) extends between bridging node 138 and the one end of the sleeve, and (ii) the other bridge (i.e., the other bridging portion of the tether) extends between the bridging node and the other end of the sleeve. For example, the bridging portions of the tether can extend from out of open ends of the lumen of the sleeve. Optionally, the bridging portions of the tether can extend laterally out through the wall of the sleeve, e.g., from sites that are closer together than the ends of the sleeve.

Connectors 142 of implant 110 are axially contractible, e.g., in response to pulling of tethers 130. That is, connectors 142 become shorter as tethers 130 are tensioned, and slide beyond the terminal anchors of the anchoring assemblies. For some applications, connectors 142′ of implant 110′ are also axially contractible. However, for some applications, connectors 142′ can be resistant to axial contraction. For example, sleeves 122 can resist compression in response to tensioning of tethers 130.

Although FIGS. 1A-2D, and 5 show valve 8 as the mitral valve of the heart, and system 100, implant 110, and implant 110′ being used at the mitral valve, system 100 and these implants can similarly be used at the tricuspid valve of the heart.

Reference is made to FIGS. 7A-B, 8A-G, 9A-B, and 10, which are schematic illustrations of a system 200 for use at atrioventricular valve 8, and techniques for use with the system, in accordance with some applications. System 200 comprises an implant 210, and often further comprises a delivery instrument 250 that is transluminally advanceable to the heart, and that is used to facilitate implantation of implant 210. Delivery instrument 250 often comprises a driver 260, and a catheter (e.g., a flexible tube) 270 within (e.g., through) which the driver is slidable.

FIG. 7A shows implant 210 in its extended state, e.g., after implantation at valve 8. FIG. 7B shows implant 210 in its contracted state, toward which the implant is subsequently transitioned in order to reshape tissue—e.g., the valve annulus. For clarity, FIGS. 7A-B do not show valve 8 or other anatomy.

At least once implanted, implant 110 comprises (i) at least two anchoring assemblies 240, each of which comprises multiple anchors 220 and a connector 242 that connects the multiple anchors to each other, and (ii) one or more bridges 244 that couple the anchoring assemblies to each other. Often, each bridge 244 is coupled to at least one other bridge at a bridging node 238. Often, at each bridging node, implant 210 comprises a respective adjustment mechanism 230, described hereinbelow. In the application shown in FIGS. 7A-9B, implant 210 has exactly two anchoring assemblies 240, exactly four bridges 244, which are coupled to each other in pairs at exactly two bridging nodes 238, with an adjustment mechanism 230 at each of the bridging nodes. FIG. 10 shows another application, designated implant 210′, which comprises only two bridges 244 coupled to each other at one bridging node 238.

Driver 260 often comprises a flexible stem 262, and a driver head 264 that is configured to reversibly engage a driver interface (e.g., defined by head 222) of anchor 220. Components and/or features of delivery instrument 150 (e.g., catheter 170 and/or driver 160) and/or anchors 120 can share one or more structural and/or functional characteristics described in one or more of the following references (e.g., structural and/or functional characteristics of one or more of the catheters, drivers, and/or anchors described therein), each of which is incorporated herein by reference:
US Patent Application Publication 2018/0049875 to Iflah et al.
PCT application publication WO/2020/240282 to Brauon et al.
U.S. patent application Ser. No. 17/145,258 to Kasher et al.

For some applications, implant 210 is transluminally delivered with anchors 220 already coupled to connectors 242. For example, and as shown, each anchor 220 can be rotatably coupled to a connector 242, with a head 222 of the anchor on one side of the connector, a tissue-engaging element 224 of the anchor on the other side of the connector, and a neck 226 of the anchor extending through the connector. For such applications, driver 260 can anchor each anchoring assembly 240 to tissue 10 by screwing tissue-engaging element 224 into the tissue by applying torque to head 222, e.g., such that neck 226 rotates freely within connector 242.

Optionally, anchors 220 can be transluminally delivered independently of implant 110. For example, once implant 110 is disposed in the atrium, each anchor 220 can be advanced to the implant and driven through connector 242 and into tissue 10.

FIGS. 8A-G show at least some steps of a technique for use with implant 210, in accordance with some applications. FIG. 8A shows implant 210 having been delivered to the atrium, and placed on the upstream side of valve 8, and tool 260 being used to anchor one of anchors 220 to tissue 10. FIGS. 8B-D show tool 260 (e.g., driver head 264 thereof) being moved successively to the other anchors 220 and anchoring them to the tissue.

The anchors 220 of one anchoring assembly 240 can be considered to be anchored in a first row along the tissue of the heart, and the anchors of the other anchoring assembly can be considered to be anchored in a second row along the tissue of the heart, across valve 8 from the first row. In the example shown, each anchoring assembly 240 comprises two anchors 220, but a greater number of anchors per anchoring assembly can be used.

Each bridging node 238 and each adjustment mechanism 230 can be partway between first and second rows of anchors (e.g., partway between anchoring assemblies 240). Often, implant 210 is implanted such that each bridging node 238 and each adjustment mechanism 230 is disposed over the upstream side of the orifice and/or a leaflet of valve 8.

Once implant 210 has been anchored, at least one tool 280 is subsequently transluminally advanced to the heart, e.g., via catheter 270, until the tool engages adjustment mechanism 230 (FIG. 8F). Tool 280 often engages adjustment element 230 within the atrium, such as over the orifice and/or leaflets of valve 8. Often, this engagement occurs between anchoring assemblies 240, exclusive. Tool 280 is then used to actuate adjustment mechanism 230 (described hereinbelow), which shortens bridges 244 (bridging length d2 in FIG. 7B being shorter than bridging length d1 in FIG. 7A), thereby drawing anchoring assemblies 240, and the tissue portions to which they are anchored, toward each other (FIG. 8G).

In the application shown, two tools 280 are engaged with a respective two adjustment mechanisms 230 in parallel, e.g., allowing simultaneous and/or back-and-forth adjustment of the two adjustment mechanisms. However, for some applications a single tool is used to engage and adjust each adjustment mechanism 230 in turn. In either case, implant 210 allows each adjustment mechanism to be adjusted independently, thereby facilitating differential contraction of different portions of the valve annulus.

For some applications, and as shown, each bridge 244 comprises a first bridge component 244a and a second bridge component 244b, which are couple to each other in a manner that facilitates axial sliding with respect to each other. For example, and as shown, one of the bridge components (bridge component 244a in the example shown) can define an axial slot 238 or groove, and the other of the bridge components (bridge component 244b in the example shown) can define a bearing that protrudes into (e.g., though) the slot or groove. For such applications, actuation of adjustment mechanism 230 adjusts the bridging length of the bridges 244 to which it is coupled by sliding bridge components 244a and 244b with respect to each other. For some applications, this configuration of the bridge components with respect to each other can be considered a telescopic arrangement.

For some applications, for each bridge 244, in addition to the axial sliding described hereinabove, bridge component 244a is articulatable with respect to bridge component 244b. For some applications, bridge component 244a is not articulatable with respect to bridge component 244b—e.g., the bridge components are movable with respect to each other only by axial sliding.

For some applications, and as shown, the coupling between each bridge 244 (e.g., each bridge component 244b) and anchoring assembly 240 (e.g., connector 242) is an articulated coupling.

For some applications, and as shown, at each bridging node 238 a respective adjustment mechanism 238 is coupled to two respective bridges 244 (e.g., the adjustment mechanism couples the two bridges together). For some applications, this coupling is such that actuation of the adjustment mechanism adjusts the bridging length of both bridges. However, for other such applications, this coupling is such that actuation of the adjustment mechanism adjusts the bridging length of only one of the bridges—e.g., one of the bridges having an adjustable bridging length, and one of the bridges having a fixed bridging length.

For some applications, adjustment mechanism 230 is operatively coupled to bridges 244 such that movement of an element, such as a bolt 232, of the adjustment mechanism along an axis ax1 causes contraction of bridges 244 (e.g., sliding of bridge component 244b) along an axis ax2 that is orthogonal to axis ax1. As illustrated, for some applications, this is achieved by operatively coupling (1) the sliding coupling of bridge element 244b with respect to bridge element 244a, with (2) a linker 236 that is articulatably coupled (i) at one end to bolt 232, and (ii) at the other end to bridge element 244b. For some applications, this arrangement is a Scott Russell linkage.

For some applications, adjustment mechanism 230 comprises a rotatable member, and is actuatable by rotating the rotatable member. For some such applications, the rotatable member is bolt 232. For example, bolt 232 can be a threaded bolt, mounted in a mount 234, such that rotation of the rotatable member moves the rotatable member along axis ax1 with respect to mount 234 (and often with respect to bridge component 244a), thereby contracting bridges 244 medially. Mount 234 can be fixedly coupled to bridge element 244a.

Although FIGS. 8A-G show valve 8 as the mitral valve of the heart, and system 200 and implant 210 being used at the mitral valve, system 200 and implant 210 (as well as implant 210′, described hereinbelow) can similarly be used at the tricuspid valve of the heart. FIGS. 9A-B show an example of this, in which valve 8 is the tricuspid valve. FIG. 9A shows implant 210 after it has been anchored to the annulus of the tricuspid valve, and FIG. 9B shows the implant after its subsequent adjustment, with the resulting contraction of the tricuspid valve.

For some applications, and as shown for implant 210, the connectors of the implant (e.g., connectors 242) are generally straight, and anchors 220 are anchored in a generally straight line. For some applications, and as shown for implant 210, the connectors of the implant (e.g., connectors 242′) are curved, and anchors 220 are anchored in an arc.

Implant 210 is shown as comprising two anchors 220 per anchoring assembly 240, and implant 210′ is shown as comprising three anchors per anchoring assembly. It is to be understood that for some applications each anchoring assembly of implants 210 or 210′ can comprise a greater number of anchors. Furthermore, for some applications, implant 210 or 210′ can comprise more anchors in one anchoring assembly than in another (e.g., the other) anchoring assembly. For example, one anchoring assembly can have two anchors, and the other anchoring assembly can have three anchors.

For some applications, connectors 242 and/or 242′ are rigid. For some applications they are semi-rigid. For some applications they are flexible. For some applications, connectors 242 and/or 242′ are non-isometrically flexible—e.g., more flexible in an atrioventricular direction than in a mediolateral direction, such as to allow the connectors to adapt to the topography of the annulus while retaining the ability to pull the annulus medially upon contraction.

For some applications, bridges 244, the coupling of the bridges to anchoring assemblies 240, and/or the coupling of the bridges to adjustment mechanism 230, are rigid in at least one plane, e.g., in a plane on which connectors 242 or 242′ lie. For example, and as shown, bridges 244 can comprise one or more rigid components, and/or can be articulatably (e.g., hingedly) coupled to the adjustment mechanism and/or to the connectors.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the treatment techniques, methods, operations, steps, etc. described or suggested herein can be performed on a living animal or on a non-living simulation, such as on a cadaver, cadaver heart, simulator (e.g., with the body parts, tissue, etc. being simulated), etc.

What is claimed is:

1. A system for use at a valve disposed between an atrium and a ventricle of a heart of a subject, the system comprising:
a catheter, transluminally advanceable to the heart;
an implant, deployable from the catheter within the heart, and comprising:
a first anchoring assembly and a second anchoring assembly, each comprising:
multiple anchors, and
a connector, connecting the multiple anchors to each other;
a bridge, coupling the first anchoring assembly to the second anchoring assembly, and having an adjustable bridging length;
a driver, extendable through the catheter, and configured to use the anchors of the first anchoring assembly to anchor the first anchoring assembly, within the atrium, at a first side of the valve, and to use the anchors of the second anchoring assembly to anchor the second anchoring assembly, within the atrium, at a second side of the valve, such that the bridge spans at least partway across the valve; and
a tool, transluminally advanceable to the heart, and configured to adjust a distance between the first anchoring assembly and the second anchoring assembly by adjusting the bridging length of the bridge.

2. The system according to claim 1, wherein the valve has an annulus and leaflets, the annulus circumscribing an orifice within which the leaflets are disposed, and wherein the tool is configured to engage the implant, within the atrium, over the orifice.

3. The system according to claim 1, wherein the valve has an annulus, and wherein the tool is configured to contract the annulus by adjusting the bridging length.

4. The system according to claim 1, wherein the valve has an annulus, a first leaflet, and a second leaflet, the annulus circumscribing an orifice within which the first and second leaflets are disposed, and wherein the tool is configured to reshape the annulus by adjusting the bridging length in a manner that draws the first leaflet and the second leaflet towards each other.

5. The system according to claim 1, wherein the tool is configured to engage the implant between the first anchoring assembly and the second anchoring assembly, exclusive.

6. The system according to claim 1, wherein the connector of each of the anchoring assemblies is rigid.

7. The system according to claim 1, wherein the connector of each of the anchoring assemblies is semi-rigid.

8. The system according to claim 1, wherein the connector of each of the anchoring assemblies is flexible.

9. The system according to claim 1, wherein the connector of each of the anchoring assemblies is non-isometrically flexible.

10. The system according to claim 1, wherein the connector of each of the anchoring assemblies comprises a polymer.

11. The system according to claim 1, wherein the connector of each of the anchoring assemblies is axially contractible.

12. The system according to claim 1, wherein the connector of each of the anchoring assemblies is resistant to axial contraction.

13. The system according to claim 1, wherein the bridge is articulatably coupled to one of the first and second anchoring assemblies.

14. The system according to claim 1, wherein the bridge is slidably coupled to one of the first and second anchoring assemblies.

15. The system according to claim 1, wherein:
the bridge is a first bridge, extending from a first part of the first anchoring assembly; and
the implant further comprises a second bridge, extending from a second part of the first anchoring assembly.

16. The system according to claim 1, wherein:
the bridge comprises first and second bridge components axially slidable with respect to each other, and
the implant further comprises one or more adjustment mechanisms, each of the adjustment mechanisms being operatively coupled to the bridge such that, for each of the adjustment mechanisms, actuation of the adjustment mechanism adjusts the bridging length of the bridge by sliding the first and second bridge components of the bridge with respect to each other.

17. The system according to claim 1, wherein the bridge is a first bridge of one or more bridges of the implant, and wherein the implant further comprises one or more adjustment mechanisms, each of the adjustment mechanisms being operatively coupled to at least one of the bridges, such that, for each of the adjustment mechanisms, movement of an element of the adjustment mechanism along a first axis causes contraction of the at least one of the bridges along a second axis, the second axis being orthogonal to the first axis.

18. The system according to claim 1, wherein the bridge is a first bridge of one or more bridges of the implant, and wherein the implant further comprises one or more adjustment mechanisms, each of the adjustment mechanisms being operatively coupled, in a Scott Russell linkage, to at least one of the bridges.

19. The system according to claim 1, wherein the tool is transluminally advanceable to the implant, and is configured to engage the implant within the atrium.

20. The system according to claim 1, wherein the bridge comprises a tether.

21. The system according to claim 20, wherein adjusting the bridging length of the tether comprises adjusting tension in the tether.

22. The system according to claim 21, further comprising a lock, the tool being configured to lock the tension in the tether by locking the lock to the tether.

23. The system according to claim 20, wherein adjusting the bridging length of the tether comprises tensioning the tether.

24. The system according to claim 1, wherein:
the bridge is a first bridge,
the implant further comprises:
a third anchoring assembly, comprising:
multiple anchors, and
a connector that connect the multiple anchors of the third anchoring assembly to each other; and
a second bridge, coupling the third anchoring assembly to an anchoring assembly selected from the group of: the first anchoring assembly and the second anchoring assembly, and having an adjustable bridging length, and
the tool is further configured to adjust a distance between the first anchoring assembly and the selected anchoring assembly by adjusting the bridging length of the second bridge.

25. A system for use at a valve disposed between an atrium and a ventricle of a heart of a subject, the system comprising:
a catheter, transluminally advanceable to the heart;
an implant, deployable from the catheter within the heart, and comprising:
multiple anchoring assemblies, each of the anchoring assemblies comprising:
multiple anchors, and
a connector, connecting the multiple anchors of the anchoring assembly to each other;
multiple bridges, coupling the anchoring assemblies to each other, each of the bridges having an adjustable bridging length;
a driver, extendable through the catheter, and configured to anchor the implant within the atrium by, for each of the anchoring assemblies, using the anchors of the anchoring assembly to anchor the anchoring assembly within the atrium, such that the bridges span, between the anchoring assemblies, at least partway across the valve; and
a tool, transluminally advanceable to the heart, and configured to adjust a distance between the first anchoring assembly and the second anchoring assembly by adjusting the bridging length of the bridge.

26. The system according to claim 25, wherein:
the multiple anchoring assemblies comprise:
a first anchoring assembly, and
one or more other anchoring assemblies, and
each of the multiple bridges couples the first anchoring assembly to one of the one or more other anchoring assemblies.
